(19) United States
(12) Patent Application Publication
(10) Pub. No.: US 2018/0099102 A1
(43) Pub. Date: Apr. 12, 2018

(54) SAFETY SYRINGE STRUCTURE

(71) Applicant: WING YOUNG CO., LTD., TAOYUAN CITY (TW)

(72) Inventors: HSIEN CHENG CHEN, TAOYUAN CITY (TW); MAO-PANG CHEN, TAOYUAN CITY (TW); WEI-LUN CHEN, TAOYUAN CITY (TW)

(21) Appl. No.: 15/287,724

(22) Filed: Oct. 6, 2016

(51) Int. Cl. A61M 5/32; A61B 5/15; A61M 5/50

(52) U.S. Cl. CPC A61M 5/3271; A61M 5/50; A61B 5/150633; A61M 5/3204

(57) ABSTRACT

A safety syringe structure includes a plunger having two ends respectively forming first and second push sections. The first push section is provided with a cover that includes a peg extending therefrom. The peg has a distal end forming a retention element and is combinable with a connection member that has an opposite end forming a hollow tube. The connection member includes a constraint hole formed therein and in communication with the outside. The constraint hole includes a stepped section formed therein. The peg includes an elastic element fit thereto and is insertable into the constraint hole such that the retention element is in abutting engagement with the stepped section. A barrel that has an interior forming a receiving space has a top end face forming flanges extending therefrom and a bottom end face on which a hollow mounting section is mounted for receiving a needle mounted thereto.

### TECHNICAL FIELD OF THE INVENTION

The present invention relates generally to a structure of a syringe, and more particularly to a safety syringe structure that allows a tube to cover a needle after injection so as to improve efficiency and safety.

### DESCRIPTION OF THE PRIOR ART

Injection of medicine into human body with a syringe is considered an invasive medical treatment, where a needle must be inserted into the human body. The needle so used would carry body fluid on or inside the needle. This is the primary reason that medical personnel or medical waste processing personnel may easily get infected of diseases, particularly blood borne diseases, such as acquired immune deficiency syndrome (AIDS) and tuberculosis (TB), due to being punctured by the used syringe needle that is improperly or carelessly processed after the administration of an injection.

A syringe is often used in combination with a cap arranged to house a needle of the syringe for the purposes of protecting the needle from damage and contamination before use of the needle and also for covering the needle with the cap after the use of the syringe to prevent undesired puncture of medical personnel or medical waste processing personnel in order to avoid occurrence of infection of the medical personnel or the waste processing personnel. However, when a person attempts to cover the used needle with the cap, due to the small size of the opening of the needle cap, it is often difficult to correctly insert the needle into the cap. In case of misalignment due to incorrect insertion direction or simply due to inadvertency and carelessness, unexpected puncture by the needle may occur. This would expose people in potential risk of disease infection and spread.

Besides the above-discussed drawbacks, an additional shortcoming is that after the medical personnel has completed an injection operation, it is necessary to take out the cap and then put the cap over the needle. There is definitely room for further improvement concerning safety and efficiency.

In view of the above, the present invention aims to provide a solution that overcome the above discussed problems.

### SUMMARY OF THE INVENTION

In view of the above problems, the present invention provides a safety syringe structure, which comprises: a plunger, which has two ends respectively provided with a first push section and a second push section. The first push section is provided with a cover, and the cover has a bottom surface from which a peg extends. The peg has a distal end that forms a retention element and is combinable with a connection member. The connection member has an opposite end to which a tube having a hollow configuration is mounted. The connection member comprises a constraint hole formed therein and in communication with the outside. The constraint hole comprises a stepped section formed therein. The peg comprises an elastic element fit to and encompassing the peg. The peg is insertable into the constraint hole such that the retention element is in abutting engagement with the stepped section. A barrel has an interior forming a receiving space. The barrel has a top end face comprising flanges extending from opposite sides thereof. The barrel has a bottom end face on which a mounting section having a hollow configuration is mounted. The mounting section selectively receives a needle mounted thereto. The tube has a length greater than a length of the needle. The second push section of the plunger is receivable in the receiving space. The barrel is receivable in the tube.

The primary object of the safety syringe structure of the present invention is that at the time when an injection operation is completed, the tube may simultaneously cover and house the needle so that a user do not necessarily and additionally put a safety cap for the needle and thus efficiency and safety can be improved.

The foregoing objectives and summary provide only a brief introduction to the present invention. To fully appreciate these and other objects of the present invention as well as the invention itself, all of which will become apparent to those skilled in the art, the following detailed description of the invention and the claims should be read in conjunction with the accompanying drawings. Throughout the specification and drawings identical reference numerals refer to identical or similar parts.

Many other advantages and features of the present invention will become manifest to those versed in the art upon making reference to the detailed description and the accompanying sheets of drawings in which a preferred structural embodiment incorporating the principles of the present invention is shown by way of illustrative example.

### BRIEF DESCRIPTION OF THE DRAWINGS

FIG. 1 is an exploded view of the present invention.

FIG. 2 is a schematic plan view of the present invention, of which a circled portion is additionally illustrated in an enlarged form.

FIG. 3 is a schematic view of the present invention, of which a circled portion is additionally illustrated in an enlarged form, to illustrate a secured condition.

FIG. 4 is a schematic view of the present invention, of which a circled portion is additionally illustrated in an enlarged form, to illustrate a released condition.

FIG. 5 is a perspective view of the present invention, of which a circled portion is additionally illustrated in an enlarged form, to illustrate the condition shown in FIG. 4.

FIG. 6 is a perspective view illustrating a use condition of the present invention.

FIG. 7 is a schematic view illustrating an application of the present invention in performing an injection operation.

### DETAILED DESCRIPTION OF THE PREFERRED EMBODIMENTS

The following descriptions are exemplary embodiments only, and are not intended to limit the scope, applicability or configuration of the invention in any way. Rather, the following description provides a convenient illustration for implementing exemplary embodiments of the invention. Various changes to the described embodiments may be made in the function and arrangement of the elements described without departing from the scope of the invention as set forth in the appended claims.

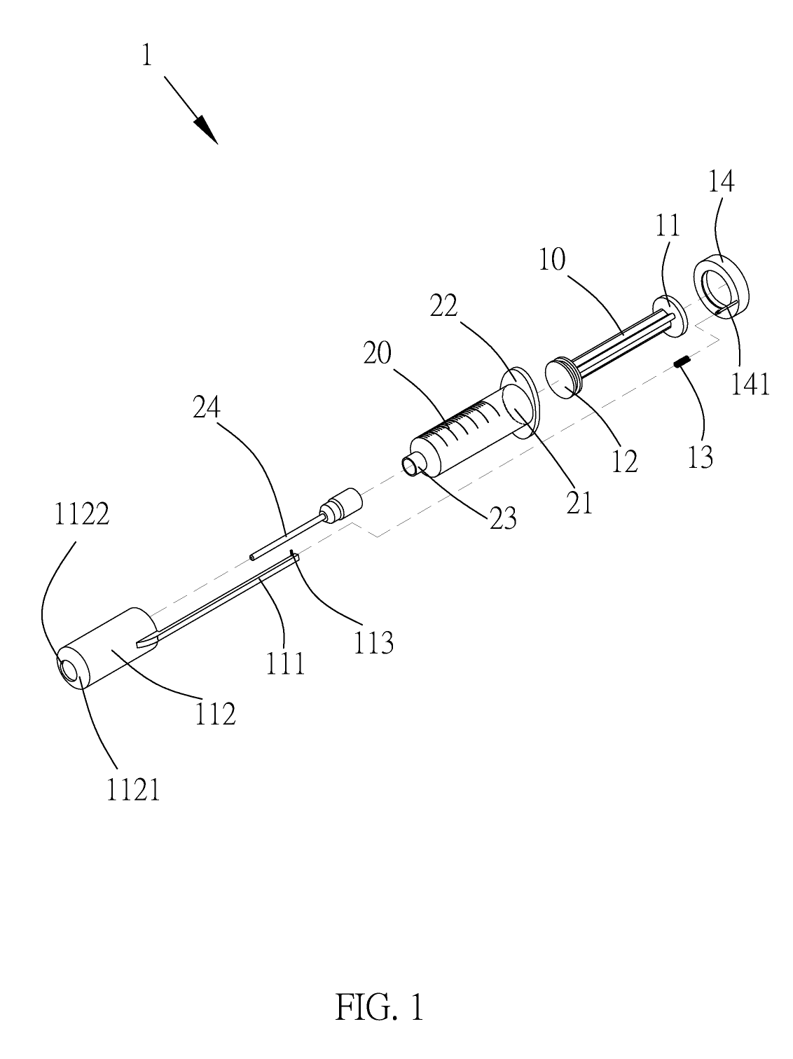

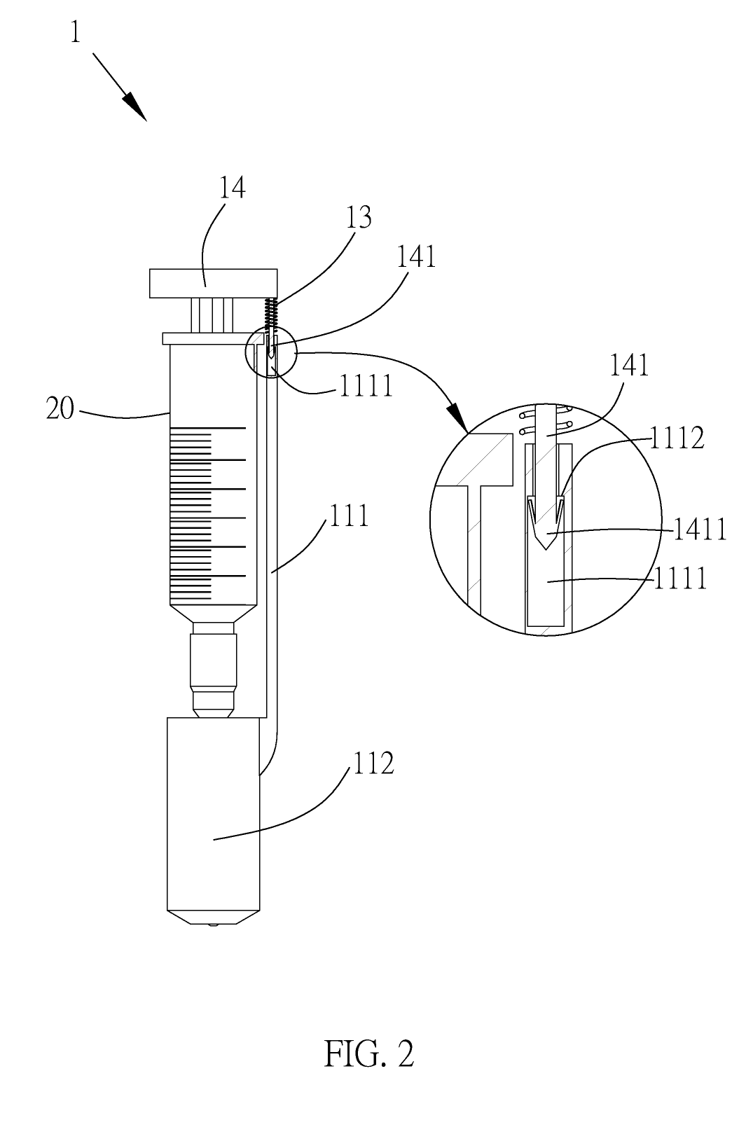

Referring to FIGS. 1 and 2, the present invention provides a safety syringe structure 1, which comprises: a primary plunger 10 and a barrel 20.

These constituent components/elements will be individually described as follows.

The plunger 10 has two opposite ends respectively provided with a first push section 11 and a second push section 12. The first push section 11 is additionally provided with a cover 14, wherein the cover has a bottom surface from which a peg 141 extends. The peg 141 comprises an elastic element 13 fit to and encompassing the peg. The peg 141 has a distal end that is provided with a retention element 1411. The peg 141 is combinable with a connection member 111. The connection member 111 comprises a pawl 113 formed thereon and projecting sideways therefrom.

The connection member 111 comprises a constraint hole 1111 formed therein and communicating with the outside. The constraint hole 1111 is provided, on an inner circumference thereof, with a stepped section 1112. The peg 141 is insertable into the constraint hole 1111 such that the retention element 1411 is in abutting engagement with the stepped section 1121 to prevent the peg 141 from detaching. Provided at an opposite end of the connection member 111 is a tube 112, which is of a hollow configuration. The tube 112 has an end face that forms a stop section 1121. The stop section 1121 is provided with a through hole 1122 formed therein to correspond to a mounting section 23 of the barrel 20.

The barrel 20 has a hollow interior forming a receiving space 21. The barrel 20 has a top end face comprising flanges 22 respectively extending sideways from opposite sides thereof. The barrel 20 has a bottom end face from which the mounting section 23, which is of a hollow configuration, extends outward. The mounting section 23 provides a function of mounting and supporting a needle 24 thereon. The tube 112 has a length that is greater that a length of the needle 24. The second push section 12 of the plunger 10 is receivable in the receiving space 21 and the barrel 20 is partly receivable in the tube 112. In the instant embodiment, the barrel 20 has a length that is greater than the length of the tube 112 and the barrel 20 has a diameter that is smaller than a diameter of the tube 112 so that the barrel 20 is receivable into the tube 112.

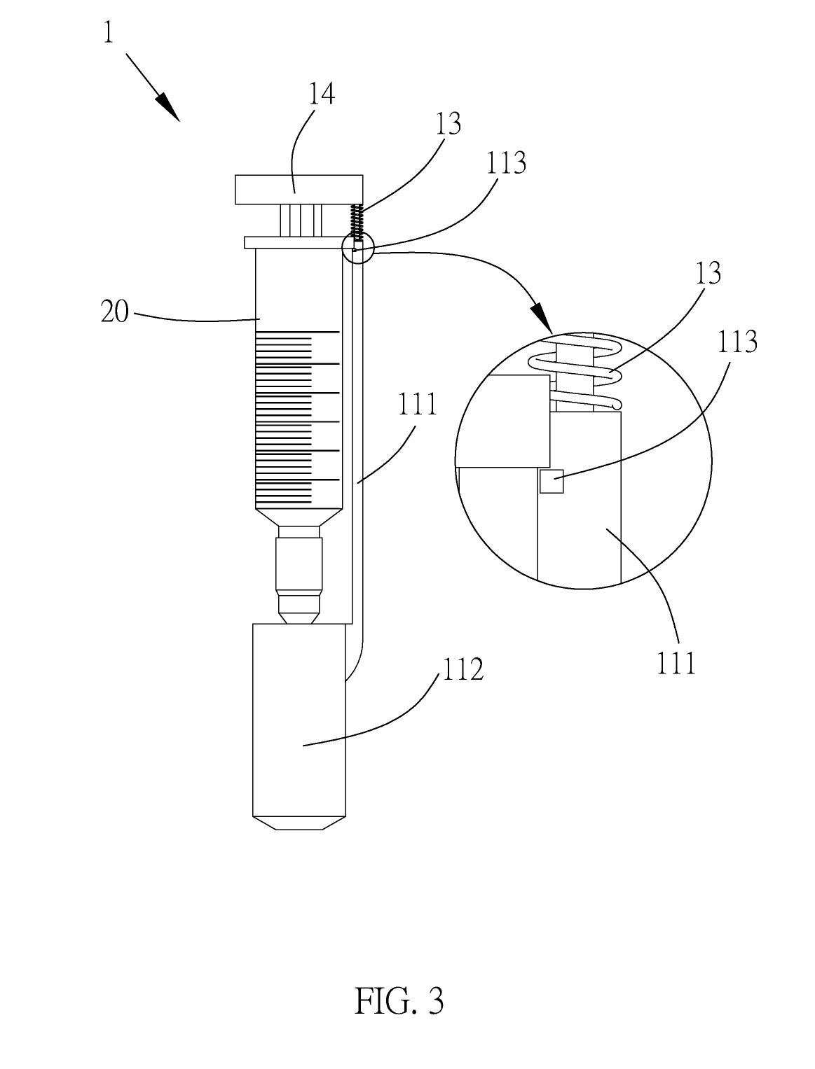

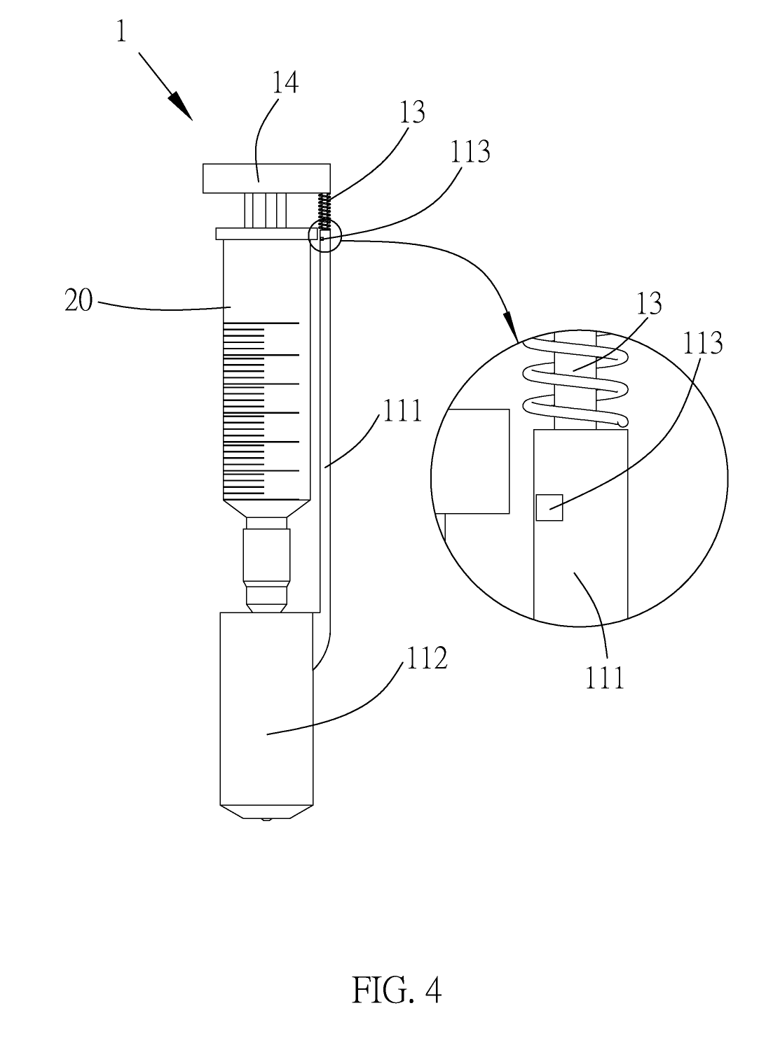

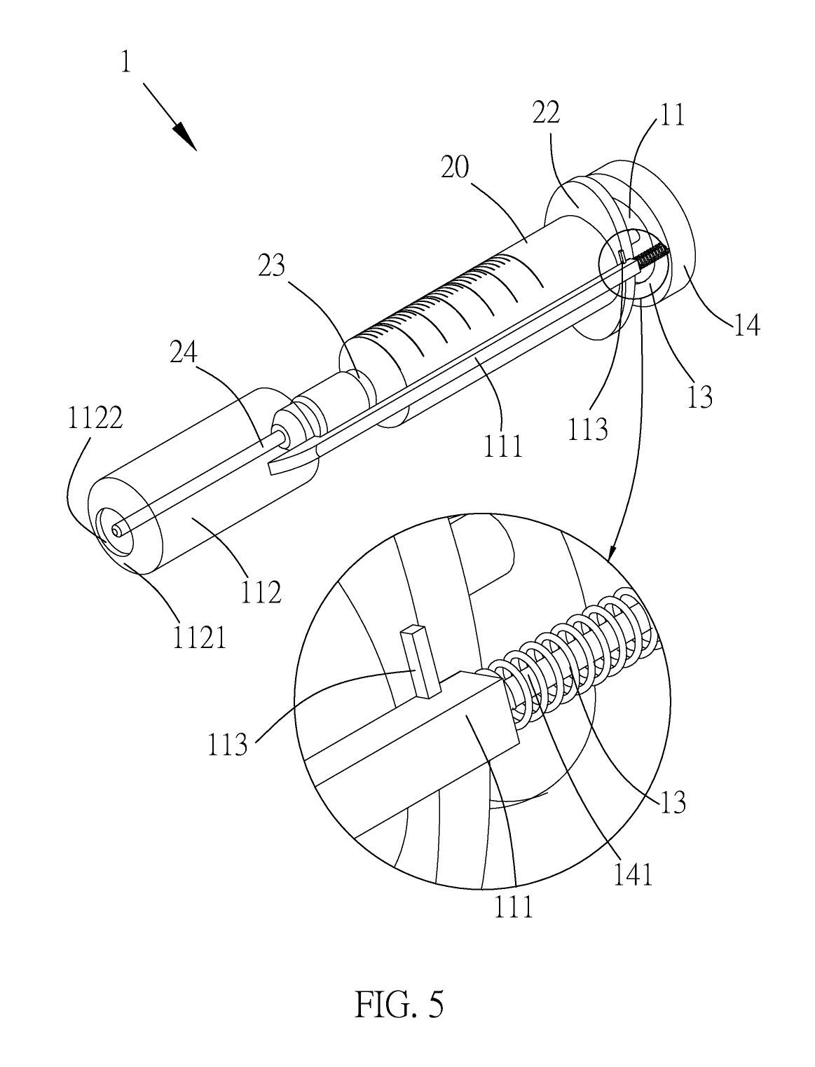

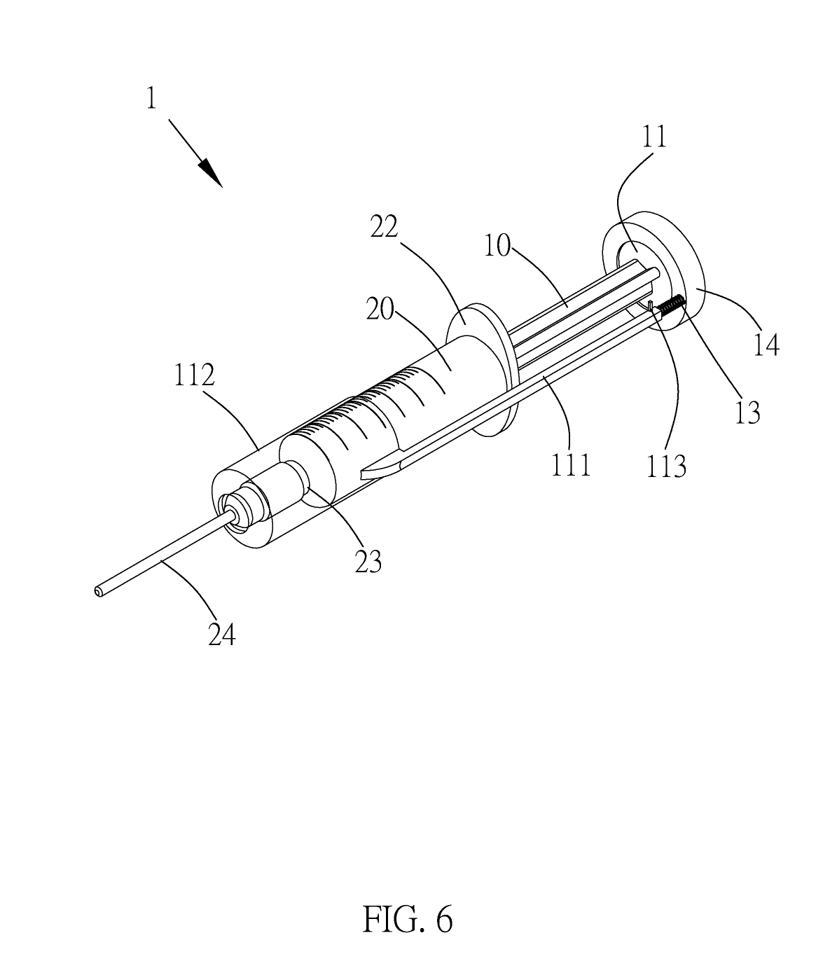

Referring to FIG. 3, in an initial condition of the safety syringe structure 1 according to the present invention, the pawl 113 is kept in engagement with and thus retaining the flange 22 in position so that the plunger 10 is fixed in position and is not allowed to move. Thus, to use, the cover 14 must be first rotated and due to the cover 14 being coupled with the first push section 11 of the plunger 10, the cover 14 and the first push section 11 are rotated in unison with each other such that the pawl 113 is moved to separate from and thus release the flange 22 (FIGS. 4 and 5). Referring to FIGS. 2 and 6, after the pawl 113 is separated from the flange 22, the plunger 10 is allowed to move in order to conduct an operation of drawing blood or taking in medicine liquid. When the plunger 10 is pulled, the retention element 1411 of the peg 141 is caused to engage and abut the stepped section 1121 so as to drive the connection member 111 to move therewith and this prevents the elastic element 13 from detaching from the constraint hole 1111 due to stretching by the force applied thereto.

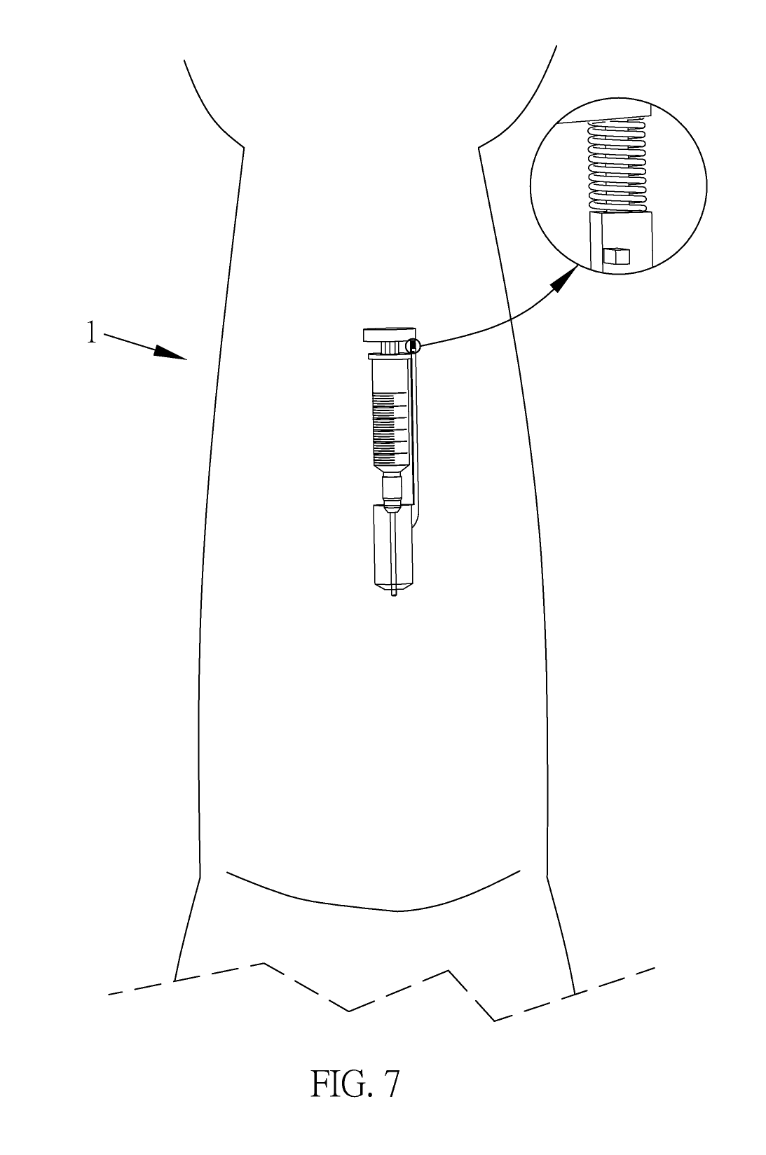

Referring to FIG. 7, to administrate an injection, the first push section 11 of the plunger 10 is pushed and the peg 141 moves deeply into the constraint hole 1111, this making the injection process stable and smooth. Further, due to the connection member 111 being coupled with the first push section 11, the tube 112 is movable in unison with the plunger 10 to gradually cover and house the needle 24. With continuous advancing of the plunger 10, the front end of the tube 112 and the stop section 1121 would get into contact with a human skin, and the elastic element 13 allows the tube 112 to retract backward for facilitating complete injection of all the medicine liquid into the human body. When the needle 24 is pulled out of the human body, the tube 112 is not blocked by the human body and spring back in the frontward direction to completely house the needle 24 inside the tube 112 thereby ensuring no undesired puncture is caused by the needle 24 and achieving the purpose of safety.

Referring to FIG. 3, after the injection, the cover 14 is rotated to have the pawl 113 engage and thus hold the flange 22 in position, restoring the condition before use, whereby the tube 112 is prevented from re-movement toward the first push section 11 to help eliminate the potential risk of being unexpectedly and accidentally punctured by the needle for greatly improving safety.

It will be understood that each of the elements described above, or two or more together may also find a useful application in other types of methods differing from the type described above.

While certain novel features of this invention have been shown and described and are pointed out in the annexed claim, it is not intended to be limited to the details above, since it will be understood that various omissions, modifications, substitutions and changes in the forms and details of the device illustrated and in its operation can be made by those skilled in the art without departing in any way from the claims of the present invention.

I claim:

1. A safety syringe structure, comprising:
a plunger, which has two opposite ends respectively provided with a first push section and a second push section, the first push section being provided with and combinable with a cover, the cover having a bottom surface from which a peg extends, the peg having a distal end that forms a retention element, the peg being combinable with a connection member, the connection member having an opposite end to which a tube having a hollow configuration is mounted, the connection member comprising a constraint hole formed therein and in communication with the outside, the constraint hole comprising a stepped section formed therein, the peg comprising an elastic element fit to and encompassing the peg, the peg being insertable into the constraint hole such that the retention element is in abutting engagement with the stepped section; and
a barrel, which has an interior forming a receiving space, the barrel having a top end face comprising a flange extending therefrom, the barrel having a bottom end face on which a mounting section having a hollow configuration is mounted, the mounting section selectively receiving a needle mounted thereto, the tube having a length greater than a length of the needle, the second push section of the plunger being receivable in the receiving space, the barrel being receivable in the tube.

2. The safety syringe structure according to claim 1, wherein the connection member of the plunger comprises a pawl formed on a lateral side thereof and projecting sideways therefrom so that the cover is rotatable to drive the connection member to rotate to such a position where the pawl is in retaining engagement with the flange.

3. The safety syringe structure according to claim 1, wherein the tube has an end face that forms a stop section, the stop section comprising a through hole formed therein to correspond to the mounting section of the barrel.

4. The safety syringe structure according to claim 1, wherein the barrel has a length greater than the length of the tube and the barrel has a diameter smaller than a diameter of the tube.
